(19) United States
(12) Patent Application Publication
(10) Pub. No.: US 2005/0107439 A1
(43) Pub. Date: May 19, 2005

(54) Composition and method for treating emesis

(76) Inventors: David Reed Helton, Foothill Ranch, CA (US); David Fick, Newport Beach, CA (US); Ernie Pfadenhauer, Cost Mesa, CA (US); Jason Sharp, San Clemente, CA (US)

Correspondence Address: SHELDON & MAK, INC, 225 SOUTH LAKE AVENUE, 9TH FLOOR, PASADENA, CA, 91101, US

(21) Appl. No.: 10/986,485

(22) Filed: Nov. 10, 2004

Related U.S. Application Data
(60) Provisional application No. 60/518,085, filed on Nov. 10, 2003.

(51) Int. Cl. A61K 31/4439; A61K 31/4192; A61K 31/4184; A61K 31/416; A61K 31/405

(52) U.S. Cl. 514/338; 514/359; 514/394; 514/406; 514/419; 514/414

(57) ABSTRACT

Methods for treating emesis using compositions comprising a bicyclic ring moiety covalently linked to a substituted arylpiperazine moiety are disclosed.

The present application claims priority from U.S. Provisional Patent Application No. 60/518,085, filed Nov. 10, 2003, the entire disclosure of which is hereby incorporated by reference.

### BACKGROUND

The treatment of emesis is a medical challenge due to the variety of different causative agents leading to emesis. While some treatments are effective in blocking emesis from one cause, the same treatments may be ineffective against emesis with a different etiology. For example, therapeutic agents that are known to be effective in blocking motion sickness have not been found to be effective against emesis that is induced chemically. Anti-emetics such as antihistamines and antimuscarinics can also produce undesirable sedative side effects.

The ineffectiveness of known anti-emetic agents against a variety of stimuli is believed to be due to the existence of different neural pathways for chemically-induced vomiting and for motion-induced emesis (see, e.g., Brand and Perry, “Drugs Used In Motion Sickness”, Pharmacol. Rev., 18:895-924 (1966)). For example, the drug xylazine, which can trigger emesis, stimulates alpha-2 noradrenergic receptors in the area postrema in the brain. Cisplatin, a cytotoxic drug compound used in cancer chemotherapy, triggers emetic effects by activating nerves passing through or close to the area postrema. Ablation of the area postrema has been found to eliminate emesis elicited by xylazine and cisplatin compounds (see, e.g., Colby et al., “Emetic Action of Xylazine on the Chemoreceptor Trigger Zone for Vomiting in Cats”, J. Vet. Pharmacol. Therap., 4:9396 (1981) and McCarthy and Borison, “Cisplatin-induced Vomiting Eliminated by Ablation of the Area Postrema in Cats”, Cancer Treat. Rep. 68:401-404 (1984)).

Motion-induced emesis, however, uses a neural pathway that does not require the area postrema. Ablation of the area postrema does not eliminate motion-induced emesis (see, e.g., Borison and Borison, “Motion Sickness Reflex Arc Bypasses the Area postrema in Cats”, Exp. Neurol., 92:723-737 (1986)).

Some agents, in particular 5-HT1A agonists, have been found to alleviate both motion- and chemical-induced emesis. Such agents produce anxiety as a side effect, however.

### SUMMARY

There remains a need for an effective treatment for emesis caused by a variety of stimuli which does not produce the unwanted side effects of presently used anti-emetic agents. The compounds and compositions disclosed herein provide such a treatment for both motion sickness and chemically-induced emesis. The present compounds and compositions do not produce the unwanted side effects, such as anxiety and sedation, of prior anti-emetic agents, or do so to a lesser extent than such agents.

### DETAILED DESCRIPTION

Definitions

As used herein, the following terms have the following meanings, unless their usage in context indicates otherwise.

The term “alkyl” refers to saturated aliphatic groups including straight-chain, branched-chain, and cyclic groups, all of which can be optionally substituted. Preferred alkyl groups contain 1 to 10 carbon atoms. Suitable alkyl groups include methyl, ethyl, and the like, and can be optionally substituted. The term “heteroalkyl” refers to carbon-containing straight-chained, branch-chained and cyclic groups, all of which can be optionally substituted, containing at least one O, N or S heteroatom. The term “alkoxy” refers to the ether —O-alkyl, where alkyl is defined as above.

The term “alkenyl” refers to unsaturated groups which contain at least one carbon-carbon double bond and includes straight-chain, branched-chain, and cyclic groups, all of which can be optionally substituted. Preferable alkenyl groups have 2 to 10 carbon atoms. The term “heteroalkenyl” refers to unsaturated groups which contain at least one carbon-carbon double bond and includes straight-chained, branch-chained and cyclic groups, all of which can be optionally substituted, containing at least one O, N or S heteroatom.

The term “aryl” refers to aromatic groups that have at least one ring having a conjugated, pi-electron system and includes carbocyclic aryl and biaryl, both of which can be optionally substituted. Preferred aryl groups have 6 to 10 carbon atoms. The term “aralkyl” refers to an alkyl group substituted with an aryl group. Suitable aralkyl groups include benzyl and the like; these groups can be optionally substituted. The term “aralkenyl” refers to an alkenyl group substituted with an aryl group. The term “heteroaryl” refers to carbon-containing 5-14 membered cyclic unsaturated radicals containing one, two, three, or four O, N, or S heteroatoms and having 6, 10, or 14 π-electrons delocalized in one or more rings, e.g., pyridine, oxazole, indole, thiazole, isoxazole, pyrazole, pyrrole, each of which can be optionally substituted as discussed above.

The term “derivative” refers to a compound that is modified or partially substituted with another component. The terms “patient,” “subject” and the like with reference to individuals that can be treated with the present compounds and/or pharmaceutical compositions refer to humans and other mammals.

The term “emesis” refers to vomiting, i.e. the reflex act of ejecting the contents of the stomach through the mouth. Included within the meaning of this term, as used herein, are emesis-related conditions, including nausea. Emesis can result from a number of different causes, including the administration of chemotherapeutic agents, motion, pregnancy (morning sickness), and viral infections.

The term “hydrocarbyl” refers to a hydrocarbon chain, which can be optionally substituted or provided with other substitutions known to the art.

The term “optionally substituted” refers to one or more substituents which can be, without limitation, alkyl, aryl, amino, hydroxy, alkoxy, aryloxy, alkylamino, arylamino, alkylthio, arylthio, or oxo, cyano, acetoxy, or halo moieties.

The term “sulfonyl” refers to the group —S(O₂)—. The term “halo” refers to fluoro-, chloro-, bromo-, or iodo-substitutions. The term “alkanoyl” refers to the group —C(O)R, where R is alkyl. The term “aroyl” refers to the group —C(O)R, where R is aryl. Similar compound radicals involving a carbonyl group and other groups are defined by analogy. The term “aminocarbonyl” refers to the group —NHC(O)—. The term “oxycarbonyl” refers to the group —OC(O)—. The term “heteroaralkyl” refers to an alkyl group substituted with a heteroaryl group. Similarly, the term “heteroaralkenyl” refers to an alkenyl group substituted with a heteroaryl group.

The terms “a,” “an,” and “the” and similar referents used herein are to be construed to cover both the singular and the plural unless their usage in context indicates otherwise. Recitation of value ranges herein is merely intended to serve as a shorthand method for referring individually to each separate value falling within the range. Unless otherwise indicated herein, each individual value is incorporated into the specification as if it were individually recited herein.

Compounds

The compounds of the present invention have the general schematic structure {A}-L-{B}, where the A moiety is a bicyclic ring structure such as tetrahydroindolone or a tetrahydroindolone derivative, L is a hydrocarbyl chain linker, and the B moiety is an arylpiperazine or arylpiperazine derivative, as described below.

The preferred compounds comprise two moieties and are believed to stimulate multiple receptors to block both motion- and chemically-induced emesis. For example, the arylpiperazine moiety (B moiety) of the present compounds is believed to affect serotonin (5-HT) and dopamine receptors. This portion of the present compounds is believed in particular to have 5-HT1 receptor agonist activity and to have activity at other receptors including, but not limited to, 5-HT1 (A-F), 5-HT2 (A-C), 5-HT3 (1-7), 5-HT4C, 5-HT5 (A-B), as well as at dopaminergic receptors including, but not limited to, D2, D3, and D4. Additionally, the tetrahydroindolone moiety (A moiety) of the present compounds is believed to have GABA activity. GABA receptors are highly localized in the hippocampal region of the brain which is associated with memory. Generally recognized GABA receptors include, but are not limited to, GABA A alpha (1-6), GABA A beta (1-3), GABA A gamma (1-3), GABA A delta, GABA A pi, GABA A theta, GABA A rho (1-3), GABA B1 (a-c), GABA B2, and GABA C. Interaction with such receptors by the present compounds is believed to have the additional benefits of treating anxiety and enhancing memory and cognition.

Bicyclic Moiety

In the compounds of the present invention, the A moiety comprises a 9 atom bicyclic moiety in which the five-membered ring has 1 to 3 nitrogen atoms and has the structure of Formula (I) below: 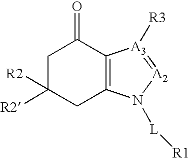
where:
(a) A₂ and A₃ are C or N;
(b) R₃ is hydrogen, alkyl, aralky, heteroaralkyl, alkenyl, aralkenyl, heteroaralkenyl, aryl, heteroaryl, or does not exist when A₃ is N;
(c) R₂ is hydrogen, alkyl, aralkyl, heteroaralkyl, aryl or heteroaryl;
(d) R_{2′} is hydrogen unless R₂ is alkyl, in which case R_{2′} is hydrogen or the same alkyl as R₂; and
(e) L and R₁ are as described below (L and R₁ are not part of the A moiety but are included in Formula (I) to show their structural relationship to the A moiety).

As shown in Formula (I), the A moiety has a six-membered saturated ring fused to a five-membered aromatic ring. The five-membered aromatic ring can have one, two or three nitrogen atoms as indicated, but the five-membered aromatic ring always has a nitrogen atom at the 1-position as indicated in Formula I. Typically, the five-membered aromatic ring has one nitrogen atom as in tetrahydroindolone. This nitrogen atom at the 1-position is covalently bonded to the linker L.

Typically the A moiety is a tetrahydroindolone moiety in which A₂ and A₃ are carbon. The tetrahydroindolone moiety can be variously substituted. One example of a tetrahydroindolone moiety for the A moiety is a tetrahydroindolone moiety of Formula (II), below: 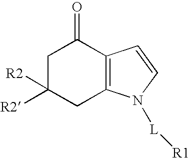
where:
(a) R₂ is hydrogen, alkyl, aralkyl, heteroaralkyl, aryl or heteroaryl;
(b) R_{2′} is hydrogen; and
(c) R₁ is as described below.

In one particularly preferred embodiment, R₂ and R_{2′}, are both hydrogen. In this particularly preferred embodiment, the A moiety is an unsubstituted tetrahydroindolone moiety.

Arylpiperazine Moiety

The B moiety, referred to above as the R₁ group, is an arylpiperazine moiety which has the structure of Formula (III) below: 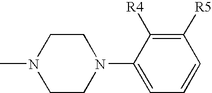
where:
(a) R₄ is hydrogen, alkyl, hydroxy, halo, alkoxy, cyano, or methylthio;
(b) R₅ is hydrogen, alkyl, hydroxy, halo, alkoxy, trifluoromethyl, nitro, amino, aminocarbonyl, or aminosulfonyl;
(c) R₄ and R₅ can be taken together to form a 5 or 6 member aromatic or non-aromatic ring, which can contain from 0 to 3 heteroatoms selected from the group of N, O, or S of which the N may be further substituted if in a non-aromatic ring.

In one embodiment, B is an m-trifluoromethylphenylpiperazinyl moiety:

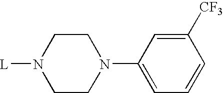

In another embodiment, B is a m-chlorophenylpiperazinyl moiety:

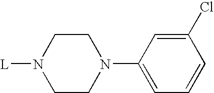

In yet another embodiment, B is an o-methoxyphenylpiperazinyl moiety:

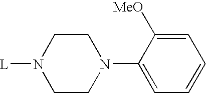

In yet another embodiment, B is a 1-naphthylpiperazinyl moiety:

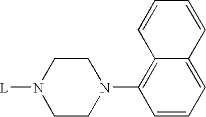

In another embodiment, the B moiety is a piperazine ring linked to a 6-member heterocyclic ring containing 1 to 3 nitrogen atoms, having the structure of Formula (IV) below:

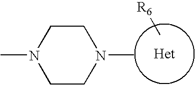

In this embodiment the 6-member heterocyclic ring (shown in Formula IV with the designation “Het”) can be, for example, a 2-pyridyl, 4-pyridyl, 2-pyrimidyl, 4-pyrimidyl, 2-pyrazinyl, or 2-triazinyl moiety. The heterocyclic ring can also be substituted, where R₆ can be a halo, alkyl, cyano, trifluoromethyl, alkoxy, amino, alkylamino, or dialkyamino group.

In one embodiment, B is a 2-pyrimidylpiperazinyl moiety:

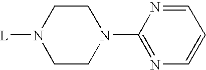

In another embodiment, the B moiety is a piperazine ring linked to a bicyclic moiety having the structure of Formula (V) below: 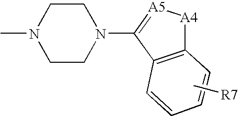
where:
(a) A₄ is N, O, or S, and when it is N, it can be further substituted with Z, which is alkyl, aralkyl, heteroaralky, or heteroalkyl.
(b) A₅ is C or N;
(c) R₇ is hydrogen, alkyl, NH₂, NHQ₁, NQ₁Q₂, OH, OQ₁, SQ₁, halo, nitro, cyano, or trifluoromethyl, where Q₁ and Q₂ are alkyl, aralkyl, heteroaralkyl, aryl, heteroaryl, alkanoyl, aroyl, aralkanoyl, heteroaralkanoyl, heteroaroyl, alkylsulfonyl, arylsulfonyl, heteroarylsulfonyl, aralkylsulfonyl, or heteroaralkylsulfonyl. The alkyl portions of Q₁ and Q₂ can be cyclic and can contain from 1 to 3 heteroatoms that can be N, O, or S. When Q₁ and Q₂ are present together and are alkyl, they can be taken together to form a 5- or 6-membered ring which may contain 1 other heteroatom which can be N, O, or S, of which the N may be further substituted with Y₂, where Y₂ is alkyl, aryl, heteroaryl, aralkyl, heteroaralkyl, alkanoyl, aroyl, heteroaroyl, aralkanoyl, heteroaralkanoyl, alkylsulfonyl, arylsulfonyl, heteroarylsulfonyl, aralkylsulfonyl, heteroaralkylsulfonyl, alkoxycarbonyl, aryloxycarbonyl, heteroaryloxycarbonyl, aralkoxycarbonyl, heteroaralkoxycarbonyl, alkylaminocarbonyl, arylaminocarbonyl, heteroarylaminocarbonyl, aralkylaminocarbonyl, or heteroaralkylaminocarbonyl. The alkyl portions of Y₂ can be cyclic and can contain from 1 to 3 heteroatoms which can be N, O, or S.

In one embodiment, B is a 3-indazolylpiperazinyl moiety: 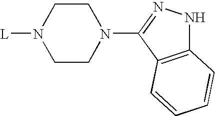
Linker Moiety

The linker moiety (L) used in the compounds of the present invention can be a hydrocarbyl chain, which can be optionally substituted as described above or substituted with other functional groups or moieties as known to those of skill in the art. Other linkers known to the art can also be used. In one embodiment, the linker moiety is a straight chain alkyl group of the formula —(CH₂)ₘ—, where m is an integer from 1 to 6 and more preferably is either 3, 4, or 5. Alternatively, the linker can be an alkyl substituted hydrocarbyl moiety of the following formula (VI): 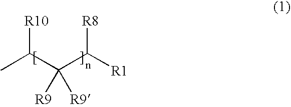
where:
(i) n is 0, 1 or 2;
(ii) R8 and R10 are hydrogen, methyl or ethyl;
(iii) R9 and R9′ are both hydrogen, methyl or ethyl;
(iv) if n is 1 and R8 or R10 is methyl or ethyl, then R9 and R9′ are hydrogen;
(v) if n is 1 and R8 and R10 are hydrogen, then R9 and R9′ are methyl or ethyl; and
(vi) if n is 2, then R9 and R9′ are hydrogen and one or both of R8 and R10 are methyl or ethyl.

The linker moiety can modulate properties of the present compounds. For example, a straight chain alkyl linker comprising two carbon atoms would provide a more rigid linkage than a longer alkyl linker. Such rigidity can produce greater specificity in target binding, while a less rigid linker moiety can produce greater potency. The solubility characteristics of the present compounds can also be affected by the nature of the linker moiety.

The use of a linker according to formula (VI) above is believed to provide a more rigid linkage compared to a straight chain linker moiety with the same number of carbon atoms in the chain. This allows for further control over the properties of the present compounds.

Generally, any A moiety can be combined with any linker L and any B moiety as described herein to produce a compound according to the present invention. However, in one embodiment the present compounds have the structure of Formula (VII) below: 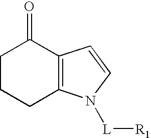
where:
(a) L is —(CH₂)ₘ— wherein m is an integer from 1 to 6; and
(b) R₁ is: 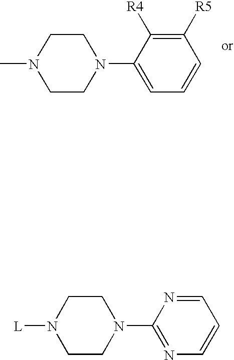
and
(c) R₄ and R₅ are the same or independently hydrogen, alkyl, hydroxy, halo, alkoxy, trifluoromethyl, nitro, amino, aminocarbonyl, or aminosulfonyl.

The compounds of the present invention further include, but are not limited to, the following compounds:

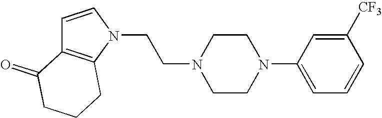

### 1-{2-[4-(3-Trifluoromethylphenyl)piperazine-1-yl]ethyl}-1,5,6,7-tetrahydroindol-4-one;

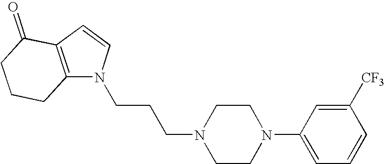

### 1-{3-[4-(3-Trifluoromethylphenyl)piperazin-1-yl]propyl}-1,5,6,7-tetrahydroindol-4-one;

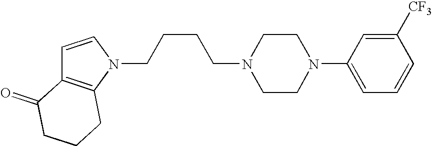

### 1-{4-[4-(3-Trifluoromethylphenyl)piperazin-1-yl]butyl}-1,5,6,7-tetrahydroindol-4-one;

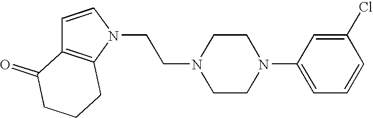

### 1-{2-[4-(3-Chlorophenyl)piperazin-1-yl]ethyl}-1,5,6,7-tetrahydroindol-4-one;

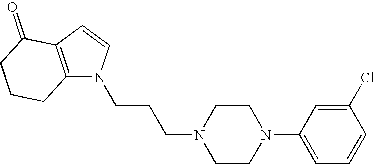

### 1-{3-[4-(3-Chlorophenyl)piperazine-1-yl]propyl}-1,5,6,7-tetrahydroindol-4-one;

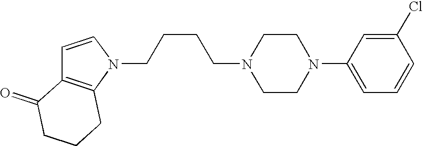

### 1-{4-[4-(3-Chlorophenyl)piperazin-1-yl]butyl}-1,5,6,7-tetrahydroindol-4-one;

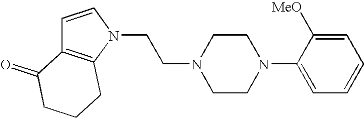

### 1-{2-[4-(2-Methoxyphenyl)piperazin-1-yl]ethyl}-1,5,6,7-tetrahydroindol-4-one;

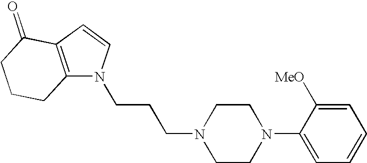

### 1-{3-[4-(2-Methoxyphenyl)piperazine-1-yl]propyl}-1,5,6,7-tetrahydroindol-4-one;

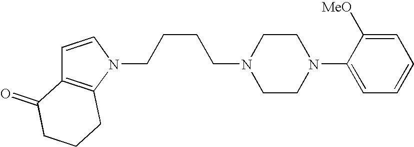

### 1-{4-[4-(2-Methoxyphenyl)piperazin-1-yl]butyl}-1,5,6,7-tetrahydroindol-4-one;

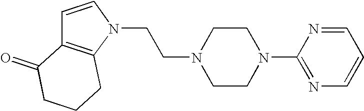

### 1-{2-[4-(2-Pyrimidyl)piperazine-1-yl]ethyl}-1,5,6,7-tetrahydroindol-4-one;

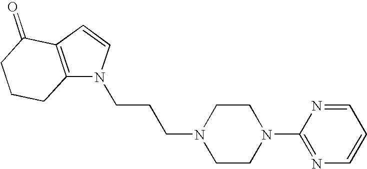

### 1-{3-[4-(2-Pyrimidyl)piperazin-1-yl]propyl}-1,5,6,7-tetrahydroindol-4-one;

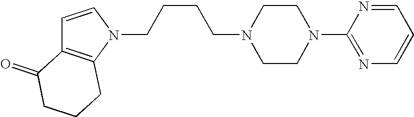

### 1-{4-[4-(2-Pyrimidyl)piperazin-1-yl]butyl}-1,5,6,7-tetrahydroindol-4-one;

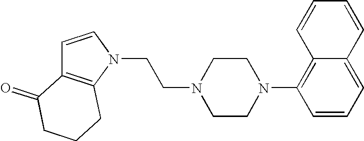

### 1-{2-[4-(1-Naphthyl)piperazin-1-yl]ethyl}-1,5,6,7-tetrahydroindol-4-one;

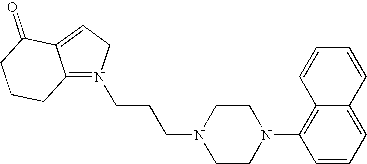

### 1-{3-[4-(1-Naphthyl)piperazin-1-yl]propyl}-1,5,6,7-tetrahydroindol-4-one;

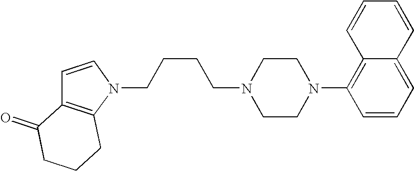

### 1-{4-[4-(1-Naphthyl)piperazin-1-yl]butyl}-1,5,6,7-tetrahydroindol-4-one;

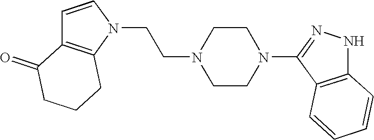

### 1-{2-[4-(3-Indazolyl)piperazin-1-yl]ethyl}-1,5,6,7-tetrahydroindol-4-one;

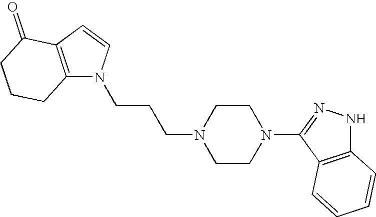

### 1-{3-[4-(3-Indazolyl)piperazin-1-yl]propyl}-1,5,6,7-tetrahydroindol-4-one; and

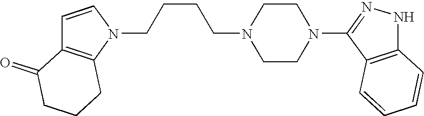

### 1-{4-[4-(3-Indazolyl)piperazin-1-yl]butyl}-1,5,6,7-tetrahydroindol-4-one

Compound Properties

Preferred compounds of the present invention have a logP of from about 1 to about 4 to enhance bioavailability and, when desired, central nervous system (CNS) penetration. Using this guideline, one of ordinary skill in the art can choose the appropriate arylpiperazine moieties to use in combination with a particular A moiety in order to ensure the bioavailability and CNS penetration of a compound of the present invention. For example, if a highly hydrophobic A moiety is chosen, with particularly hydrophobic substituents, then a more hydrophilic arylpiperazine moiety can be used.

A number of the present compounds are optically active, owing to the presence of chiral carbons or other centers of asymmetry. All of the possible enantiomers or diastereoisomers of such compounds are included herein unless otherwise indicated despite possible differences in activity.

In general, the present compounds also include salts and prodrug esters of the compounds described herein. It is well known that organic compounds, including substituted tetrahydroindolones, arylpiperazines and other components of the present compounds, have multiple groups that can accept or donate protons, depending upon the pH of the solution in which they are present. These groups include carboxyl groups, hydroxyl groups, amino groups, sulfonic acid groups, and other groups known to be involved in acid-base reactions. The recitation of a compound in the present application includes such salt forms as occur at physiological pH or at the pH of a pharmaceutical composition unless specifically excluded.

Similarly, prodrug esters can be formed by reaction of either a carboxyl or a hydroxyl group on the compound with either an acid or an alcohol to form an ester. Typically, the acid or alcohol includes an alkyl group such as methyl, ethyl, propyl, isopropyl, butyl, isobutyl, and tertiary butyl. These groups can be substituted with substituents such as hydroxy, halo, or other substituents. Such prodrugs are well known in the art. The prodrug is converted into the active compound by hydrolysis of the ester linkage, typically by intracellular enzymes. Other suitable groups that can be used to form prodrug esters are well known in the art.

Synthesis Methods

In the synthesis of the present compounds, the A moiety is generally substituted with a linker which in turn is linked to the arylpiperazine moiety (B moiety) that completes the molecule as described above. This route comprises either the steps of:
(a) synthesizing an appropriately substituted tetrahydroindolone moiety linked to an aliphatic linker in which the linker is terminated with a halogen, and reacting the halogen intermediate with the arylpiperazine to produce the final product; or
(b) synthesizing an appropriately substituted arylpiperazine moiety linked to an aliphatic linker in which the linker is terminated with a halogen, and reacting the halogen intermediate with the tetrahydroindolone to produce the final product.

Another reaction that can be used to functionalize tetrahydroindolones is the Mitsunobu reaction. The Mitsunobu reaction is a highly versatile method for the introduction of widely varying functionality upon the tetrahydroindolone moiety, because of the wide assortment of primary alcohols that are commercially available for use in this reaction.

Compounds incorporating an arylpiperazinyl moiety can be synthesized by a dihalide substitution reaction. Suitable substitution reactions are described, e.g., in M. B. Smith & J. March, “March's Advanced Organic Chemistry: Reactions, Mechanisms, and Structure” (5^{th} ed., Wiley-Interscience, New York, 2001).

The length of the linker covalently bound to the A moiety can be varied to change the distance between the A moiety and the arylpiperazine moiety in the present compounds.

### Synthesis Examples

The following representative methods for synthesizing exemplary compounds used in the present invention are merely intended as examples. Persons having ordinary skill in the art of medicinal and/or organic chemistry will understand that other starting materials, intermediates, and reaction conditions are possible. Furthermore, it is understood that various salts and esters of these compounds are also easily made and that these salts and esters can have biological activity similar or equivalent to the parent compound. Generally, such salts have halides or organic acids as anion counterions. However, other anions can be used and are considered within the scope of the present invention.

### Example 1

### Synthesis of 1-{2-[4-(3-Trifluoromethylphenyl)piperazin-1-yl]ethyl}-1,5,6,-7-tetrahydroindol-4-one

This example demonstrates a method of preparing 1-{2-[4-(3-Trifluoromethylphenyl)piperazin-1-yl]ethyl}-1,5,6,7-tetrahydroindol-4-one by a two step procedure. Generally, the arylpiperazine moieties are prepared first, then the arylpiperazine molecules are reacted with tetrahydroindolones.

Step 1: Preparation of 1-(2-Chloroethyl)-4-(3-trifluoromethylphenyl)piperazine

To a 100 mL flask was added 4-(3-trifluoromethylphenyl)piperazine HCl (5035 mg, 18.88 mmol) and 60 mL dichloromethane. 1-Bromo-2-chloroethane (1730 μL, 20.78 mmol, 1.10 eq) was added, then triethylamine (5.25 mL, 37.7 mmol, 2.00 eq). The solution was refluxed for 9 hours, then cooled to 25° C. 100 mL of hexane was then added, and the resulting suspension was vacuum filtered. The filtrate was concentrated in vacuo and purified by column chromatography using dichloromethane as eluant resulting in an oil of 1-(2-chloroethyl)-4-(3-trifluoromethylphenyl)piperazine.

Step 2: Preparation of 1-{2-[4-(3-Trifluoromethylphenyl)piperazin-1-yl]ethyl}-1,5,6,7-tetrahydroindol-4-one

Sodium hydride (60% in oil) (85 mg, 2.1 mmol, 1.8 eq.) was added to a 10 mL pear-shaped flask. The solid was rinsed twice with 2 mL hexane to remove oil, then 3 mL anhydrous N,N-dimethylformamide (DMF) was added. 1,5,6,7-Tetrahydroindol-4-one (186.7 mg, 1.38 mmol, 1.159 eq.) was added slowly, with stirring and hydrogen evolved. The walls of the flask were washed with an additional 1 mL of anhydrous DMF. 1-(2-Chloroethyl)-4-(3-trifluoromethylphenyl)piperazine (349.00 mg, 1.19 mmol, 1.000 eq) was added as a solution in 2 mL DMF, and the mixture was stirred under nitrogen at 25 C for 8 hours. The resulting mixture was acidified with 1N HCl to pH 6, and extracted with dichloromethane. The organic layer was washed four times with 25 mL water, dried over sodium sulfate and concentrated in vacuo to an oil which was purified by column chromatography using 5% methanol in dichloromethane as eluant resulting in the title compound as an oil. The oil was dissolved in 5 mL of 50% dichloromethane in hexanes. A solution of 4N HCl in dioxane (200 L) was added and the mixture stirred for 30 minutes followed by vacuum filtration of the suspension. A white powder of the product HCl salt was recovered.

### Example 2

### Synthesis of 1-{3-[4-(3-Trifluoromethylphenyl)piperazin-1-yl]propyl}-1,5,6,7-tetrahydroindol-4-one

Step 1: Preparation of 1-(3-Chloropropyl)-4-(3-trifluoromethylphenyl)piperazine

To a 100 mL flask was added 1-(3-trifluoromethylphenyl)piperazine HCl (5035 mg, 18.88 mmol) and 60 mL dichloromethane. 1-Bromo-3-chloropropane (1730 L, 20.78 mmol, 1.10 eq) was added, then triethylamine (5.25 mL, 37.7 mmol, 2.00 eq). The solution was refluxed for 9 hours, then cooled to 25° C. 100 mL of hexane was then added, and the resulting suspension was vacuum filtered. The filtrate was concentrated in vacuo and purified by column chromatography using dichloromethane as eluant resulting in an oil of 1-(3-chloropropyl)-4-(3-trifluoromethylphenyl)piperazine.

Step 2: Preparation of 1-{2-[4-(3-Trifluoromethylphenyl)piperazin-1-yl]propyl}-1,5,6,7-tetrahydroindol-4-one

The compound is synthesized by reacting the 1-(3-chloropropyl)-4-(3-trifluoromethylphenyl) piperazine with 1,5,6,7-tetrahydroindol-4-one using step 2 of Example 1.

### Example 3

### Synthesis of 1-{3-[4-(3-Chlorophenyl)piperazine-1-yl]propyl}-1,5,6,7-tetrahydroindol-4-one

Since 1-(3-Chloropropyl)-4-(3-chlorophenyl)piperazine HCl is commercially available, step one was omitted.

To a solution of 1,5,6,7-tetrahydroindol-4-one (135 mg, 1.0 mmol) in 5 mL dimethylsulfoxide was added powdered sodium hydroxide (84 mg, 2.1 mmol) and the solution stirred for 15 minutes at 25° C. 1-(3-Chloropropyl)-4-(3-chlorophenyl)piperazine HCl (310 mg, 1.0 mmol) was then added and stirring continued overnight. Upon completion, by thin-layer chromatography (TLC), the reaction was partitioned between 50 mL each of dichloromethane and water then separated. The water layer was extracted with 50 mL more of dichloromethane and the combined organic layers washed with brine, dried with sodium sulfate, and concentrated in vacuo to dryness. The crude product was purified via flash chromatography eluting with an ethyl acetate and dichloromethane mixture resulting in the title compound as an oil. The oil was dissolved in 5 mL of 50% dichloromethane in hexanes. A solution of 4N HCl in dioxane (200 L) was added and the mixture stirred for 30 minutes followed by vacuum filtration of the suspension. A white powder of the product HCl salt was recovered.

### Example 4

### Synthesis of 1-{3-[4-(2-Methoxyphenyl)piperazine-1-yl]propyl}-1,5,6,7-tetrahydroindol-4-one

Step 1: Preparation of 1-(3-Chloropropyl)-4-(2-methoxyphenyl)piperazine

The 1-(3-Chloropropyl)-4-(3-trifluoromethylphenyl)piperazine is prepared by the same method as disclosed in step 1 of example 2 employing 1-(2-Methoxyphenyl)piperazine HCl instead.

Step 2: Preparation of 1-{3-[4-(2-Methoxyphenyl)piperazine-1-yl]propyl}-1,5,6,7-tetrahydroindol-4-one

The compound is prepared by the same method as disclosed in step 2 of example 3.

### Example 5

### Synthesis of 1-{3-[4-(2-Pyrimidyl)piperazine-1-yl]propyl}-1,5,6,7-tetrahydroindol-4-one

Step 1: Preparation of 1-(3-Chloropropyl)-4-(2-pyrimidyl)piperazine

The compound is prepared by the same method as disclosed in step 1 of example 2 employing 1-(2-Pyrimidyl)piperazine 2HCl instead.

Step 2: Preparation of 1-{3-[4-(2-Pyrimidyl)piperazine-1-yl]propyl}-1,5,6,7-tetrahydroindol-4-one

The compound is prepared by the same method as disclosed in step 2 of example 3.

### Example 6

### Synthesis of 1-{2-[4-(3-Chlorophenyl)piperazin-1-yl]ethyl}-1,5,6,7-tetrahydroindol-4-one

Step 1: Preparation of 1-(2-Chloroethyl)-4-(3-chlorophenyl)piperazine

A mixture of (3-chlorophenyl)piperazine HCl (51.5 mmol) and powdered sodium hydroxide (103 mmol) in DMSO (75 mL) was treated with 2-bromo-1-chloroethane (77.2 mmol) and stirred at ambient temperature for 4 hours. The reaction was poured into ice cold water (200 mL) and stirred for 0.5 hours. A solid mass formed and was separated by decanting the water. The aqueous layer was extracted with dichloromethane (100 mL). The solid mass was dissolved with dichloromethane (100 mL) and the combined organics were dried with sodium sulfate, filtered and the solvent removed under vacuum. Flash chromatography (chloroform:acetone 50:1 to 20:1) yielded an oil (7.95 g) as the titled compound.

Step 2:1-{2-[4-(3-Chlorophenyl)piperazin-1-yl]ethyl}-1,5,6,7-tetrahydroindol 4-one

To a solution of 1,5,6,7-tetrahyroindol-4-one (51.5 mmol) in DMSO (60 mL) was added powdered sodium hydroxide (53.9 mmol) and the mixture was stirred at ambient temperature for 0.5 hours. 1-(2-chloroethyl)-4-(3-chlorophenyl)piperazine (49.0 mmol) was then added as a solution in DMSO (20 mL) and the resulting mixture stirred at ambient temperature for 24 hours then heated to approximately 60° C. for 2 hours, after which time TLC (ethyl acetate:dichloromethane 1:1) showed complete reaction. The reaction was poured into ice cold water (300 mL) and stirred for 0.5 hours. A solid mass formed and was separated by decanting the water. The aqueous layer was extracted with dichloromethane (100 mL). The solid mass was dissolved with dichloromethane (100 mL) and the combined organics were dried with sodium sulfate and the solvent removed under vacuum. The resulting sludge was triturated with hexanes (100 mL) for 2 hours and the suspension vacuum filtered and washed with hexanes. The obtained solid was dried under vacuum resulting in a tan powder (14.57 g) as the titled compound.

### Example 7

### Synthesis of 1-{2-[4-(2-Methoxyphenyl)piperazin-1-yl]ethyl}-1,5,6,7-tetrahydroindol-4-one

Step 1: Preparation of 1-(2-Chloroethyl)-4-(2-methoxyphenyl)piperazine

A mixture of 1-(2-methoxyphenyl)piperazine HCl (52.5 mmol) and powdered sodium hydroxide (105 mmol) in DMSO (40 mL), was stirred at ambient temperature. After 0.5 hours, 1-bromo-2-chloroethane (78.8 mmol) was added to the solution and left to stir for 4 hours. The reaction was monitored by TLC (ethyl acetate: dichloromethane 1:4), upon completion, the mixture was poured into 200 mL of ice water and the product was extracted with dichloromethane twice, dried with sodium sulfate, and solvent was removed under vacuum. Flash chromatography (ethyl acetate: dichloromethane, 1:5 yielded an oil of the title compound (7.30 g).

Step 2: Preparation of 1-{2-[4-(2-Methoxyphenyl)piperazin-1-yl]ethyl}-1,5,6,7-tetrahydroindol-4-one

A mixture of 1,5,6,7-tetrahyroindol-4-one (30.1 mmol) and powdered sodium hydroxide (31.6 mmol) in DMSO (15 mL) was heated for 0.5 h, and then treated with a solution of 1-(2-chloroethyl)-4-(2-methoxyphenyl)piperazine (7.30 g) in DMSO (30 mL) dropwise. The reaction was left under heat and was monitored by TLC (ethyl acetate: dichloromethane, 1:1). After completion (˜8 hours), the reaction mixture was poured into ice water (300 mL) and extracted with dichloromethane twice, dried with sodium sulfate and the solvent removed under vacuum. Flash chromatography (ethyl acetate: dichloromethane, 1:4) yielded an oil, (7.25 g).

### Example 10

### Synthesis of 1-{4-[4-(3-Trifluoromethylphenyl)piperazin-1-yl]butyl}-1,5,6,-7-tetrahydroindol-4-one

Step 1: Synthesis of 1-(4-Chlorobutyl)-1,5,6,7-tetrahydroindol-4-one:

To a solution of 1,5,6,7-tetrahydroindol-4-one (10.0 g, 74.0 mmol) in acetone (300 mL) was added powdered sodium hydroxide (3.26 g, 81.4 mmol) and the mixture stirred at ambient temperature for 0.25 hours. 1-Bromo-4-chlorobutane (9.38 mL, 81.4 mmol) was then added and the resulting mixture stirred at ambient temperature for 7 hours after which time TLC (ethyl acetate:dichloromethane 1:1) showed complete reaction. The reaction was gravity filtered to remove salts, and the filtrate concentrated to dryness under vacuum. The resulting residue was dissolved in dichloromethane (200 mL) and gravity filtered again to remove more salts. The filtrate was then washed with water, dried with sodium sulfate, filtered and the solvent removed under vacuum to yield an oil. Flash chromatography using 6 in. of silica gel in a 5.5 cm column eluting with 1:1 followed by 2:1 ethyl acetate:hexane on half of the residue yielded 9.0 g of an oil which contained ˜6.0 g of pure product (72%) and ˜3.0 g of acetone aldol condensation product (4-hydroxy-4-methyl-2-pentanone). The oil was taken to the next step without further purification.

Step 2: Synthesis of 1-{4-[4-(3-Trifluoromethylphenyl)piperazin-1-yl]butyl}-1,5,6,7-tetrahydroindol-4-one

A mixture of 1-(4-Chlorobutyl)-1,5,6,7-tetrahydroindol-4-one (6.0 g, 26.6 mmol, as a mixture with 3.0 g of 4-hydroxy-4-methyl-2-pentanone) and sodium iodide (4.38 g, 29.2 mmol) in acetonitrile (100 mL) was heated at reflux for 6 hours. (3-Trifluoromethylphenyl)piperazine (5.81 g, 25.2 mmol) and potassium carbonate (3.67 g, 26.6 mmol) was then added and reflux continued for. 16 hours. TLC (ethyl acetate:dichloromethane 1:1) showed complete reaction. The reaction was poured into ice cold water (400 mL) and stirred for 0.5 hours. An oil separated out and was isolated from the mixture. The oil was dissolved with dichloromethane (150 mL), washed with water and brine, then dried with sodium sulfate, filtered and the solvent removed under vacuum to yield the title compound as an oil (9.7 g, 91.5%).

Preparation of Oxalate salt of 1-{4-[4-(3-Trifluoromethylphenyl)piperazin-1-yl]butyl}-1,5,6,7-tetrahydroindol-4-one. Dissolved compound (4.2 g) in hot ethyl acetate (150 mL), filtered solution hot to remove undissolved solid, and added a solution of oxalic acid (1.08 g, 1.2 eq) in methanol (10 mL) with stirring. A white precipitate formed immediately and the mixture was stirred for 0.5 hours to room temperature. Vacuum filtration and washing with ethyl acetate afforded an off-white powder upon drying (5.0 g, 98%). HPLC Purity was 98.9%.

Pre-Clinical Models of Emesis

In determining the therapeutic effects and appropriate dosages of particular compounds and pharmaceutical compositions according to the present application for a human or animal subject, animal models can be used. Exemplary animal models are set forth below. Other models known to the art can also be used.

Induction and Measurement of Chemotherapy-Induced Emesis (Emesis Model)

To test compositions for their effect on chemically-induced emesis, the following test can be performed. Male or female S. murinus (30-80 g) are maintained in a temperature-controlled room at 24±1 C under artificial lighting, with lights on between 0700 and 1730 hours. Artificial humidity is maintained at 50±5%. Animals are allowed free access to water and pelleted cat chow (e.g., Feline Diet 5003, PMI® Feeds, St. Louis, USA).

On the day of experiment, the animals are transferred to clear observation chambers (approximately 21×14×13 cm) for the assessment of emetic behavior. They are allowed 30 minutes to adapt before being injected subcutaneously with compounds or their respective vehicles. Chemotherapy emetic agents are administered intravenously following administration of test compounds. The animals are then observed for 60 minutes. An episode of emesis is characterized by rhythmic abdominal contractions that are either associated with the oral expulsion of solid or liquid material from the gastrointestinal tract (i.e. vomiting) or not associated with the passage of material (i.e. retching movements). An episode of retching and/or vomiting is considered separate when an animal changes its location in the observation chamber, or when the interval between retches and/or vomits exceeds 2 seconds.

Induction and Measurement of Motion-Induced Emesis (Emesis Model)

To test for emesis due to motion exposure, cats are placed in a transparent cage on a reciprocal shaker (e.g., Taitec, Double Shaker R-30, Taiyo Scientific Industrial Co Ltd.) after an acclimatization period of at least 5 minutes. Compounds are administered at predetermined time points before testing. The animals are exposed to horizontal motion of 4 cm displacement (2 cm left, 2 cm right) at a frequency of 1 Hz for 10 minutes. A 10 minute exposure is used to reduce the chances of obtaining a false negative result. Observation is continued for at least 5 minutes after the end of motion exposure in case a delayed response occurs, although previous studies have shown that episodes of emesis after cessation of motion are very rare.

Potentiated Startle Test (Anxiety/Adverse Effect Model)

In the fear potentiated startle model, animals are exposed to a neutral stimulus such as light (conditioned stimulus) with an aversive stimulus such as a shock (unconditioned stimulus). Following conditioning, when the animals are presented with a loud acoustic stimulus, larger startle responses are elicited when the startle stimulus is preceded by light. The difference in amplitude between the startle response when conditioned animals are exposed to the aversive stimulus paired with a neutral stimulus and the startle response when the conditioned animal is exposed only to the aversive stimulus is known as fear potentiated startle (see, e.g., Davis, TiPS, 13:35-41 (January 1992)).

Hamilton-Kinder startle chambers are used for conditioning sessions and for the production and recording of startle responses. On the first 2 days, rats are placed into dark startle chambers in which shock grids are installed. Following a 5-minute acclimation period, each rat receives a 1 mA electric shock (500 ms) preceded by a 5 second presentation of light (15 watt) which remains on for the duration of the shock. Ten presentations of the light and shock are given in each conditioning session. Rats are gavaged with a solution of test compound of water and startle testing sessions are then conducted. A block of 10 consecutive presentations of acoustic startle stimuli (110 dB, non-light-paired) are presented at the beginning of the session in order to minimize the influences of the initial rapid phase of habituation to the stimulus. This is followed by 20 alternating trials of the noise alone or noise preceded by the light. Excluding the initial trial block, startle response amplitudes for each trial type (noise-alone vs. light+noise) are averaged for each rat across the entire test session. Data are presented as the difference between noise-alone and light plus noise. Compounds that increase potentiated startle are considered to have anxiogenic activity.

Automated Elevated Plus Maze (Anxiety/Adverse Effect Model)

The Hamilton-Kinder elevated plus-maze is based on the design of Helton et al., and was originally validated for mice by Lister (1987). The maze can be made of Plexiglas having two open arms (e.g., 30×5×0.25 cm) and two enclosed arms (30×5×15 cm). The floor of each maze arm is corrugated to provide texture. The arms extend from a central platform and angled at 90 degrees from each other. The maze is elevated to a height of 45 cm above the floor and illuminated by red light. Individual infrared photocells are mounted along each arm of the maze to monitor closed, open, or nosepoke activity. Mice are individually placed on the central platform of the maze and the number of closed arm, open arm, and nosepoke (poking head only into open arm from closed arm of maze) counts are recorded and used as a measure of arm entries and time spent on various sections of the maze over a five-minute test period. An increase in closed arm activity or a decrease in open arm activity indicates anxiogenic activity of a compound. Compounds of the present invention do not show this anxiogenic activity.

Other clinically acceptable models of anxiety can also be used to determine the anxiogenic effects or dosing of a particular compound, including the Light/Dark Exploration and Maternal Separation Vocalization Tests.

Treatment of Emesis

In order to prevent or treat emesis, an effective amount of one or more of the present compounds in a pharmaceutical composition is administered to a patient in need thereof. A patient is determined to be in need of treatment with the present compounds either through observation of vomiting by the patient, or through a patient's self-reporting of emesis (in the case of a human patient). A patient is determined to be in need of preventative therapy by assessing that the patient is at risk of experiencing emesis due to another medical condition or due to exposure to an agent known to be associated with emesis, such as a viral or chemical agent or radiation.

The present compounds are beneficial in the therapy of acute, delayed or anticipatory emesis, including emesis induced by chemotherapy, radiation, toxins, viral or bacterial infections, pregnancy, vestibular disorders (e.g. motion sickness, vertigo, dizziness and Meniere's disease), surgery, migraine, and variations in intracranial pressure. The uses of this invention are of particular benefit in the therapy of emesis induced by radiation, for example during the treatment of cancer, or radiation sickness, and in the treatment of post-operative nausea and vomiting. Most especially, use of the invention is beneficial in the therapy of emesis induced by antineoplastic (cytotoxic) agents including those routinely used in cancer chemotherapy, and emesis induced by other pharmacological agents, for example, alpha-2 adrenoceptor antagonists, such as yohimbine, MK-912 and MK-467, and type IV cyclic nucleotide phosphodiesterase (PDE4) inhibitors, such as RS14203, CT-2450 and rolipram.

Particular examples of chemotherapeutic agents are described, for example, by D. J. Stewart in Nausea and Vomiting: Recent Research and Clinical Advances, ed. J. Kucharczyk et al., CRC Press Inc., Boca Raton, Fla., USA, 1991, pages 177-203, especially page 188. Commonly used chemotherapeutic agents include cisplatin, cyclophosphamide, dacarbazine (DTIC), dactinomycin, mechlorethamine (nitrogen mustard), streptozocin, cyclophosphamide, carmustine (BCNU), lomustine (CCNU), doxorubicin (adriamycin), daunorubicin, procarbazine, mitomycin, cytarabine, etoposide, methotrexate, 5-fluorouracil, vinblastine, vincristine, bleomycin, paclitaxel and chlorambucil (R. J. Gralle et al. in Cancer Treatment Reports, 1984, 68, 163-172). Emesis due to other chemical agents, such as the toxins soman or sarin, can also be prevented and/or treated.

The present compounds are administered to a patient in a quantity sufficient to treat or prevent the symptoms and/or underlying etiology associated with emesis in the patient. In a preferred embodiment, the compounds are administered prior to administration of an agent which is likely to cause emesis, such as one or more of the chemotherapeutic agents described above. The present compounds can also be administered in combination with such agents, either in physical combination or in combined therapy through the administration of the present compounds and agents in succession (in any order). Although the present invention is useful in any mammal suffering from emesis, a preferred subject is a human.

The present invention thus includes the use of the present compounds in a pharmaceutical composition to prevent and/or treat emesis. In addition, the invention includes the use of these compounds for the manufacture of a medicament for the prevention and/or treatment of emesis.

Dosing

Depending upon the particular needs of the individual subject involved, the compounds of the present invention can be administered in various doses to provide effective treatment concentrations based upon the teachings of the present invention. Factors such as the activity of the selected compounds, the physiological characteristics of the subject, the extent or nature of the subject's disease or condition, and the method of administration will determine what constitutes an effective amount of the selected compounds. Generally, initial doses will be modified to determine the optimum dosage for treatment of the particular subject. The compounds can be administered using a number of different routes including oral administration, topical administration, transdermal administration, intraperitoneal injection, or intravenous injection directly into the bloodstream. Effective amounts of the compounds can also be administered through injection into the cerebrospinal fluid or infusion directly into the brain, if desired.

An effective amount of any embodiment of the present invention is determined using methods known to pharmacologists and clinicians having ordinary skill in the art. For example, the animal models described herein can be used to determine applicable dosages for a patient. As known to those of skill in the art, a very low dose of a compound, i.e. one found to be minimally toxic in animals (e.g., 1/10×LD10 in mice), can first be administered to a patient, and if that dose is found to be safe, the patient can be treated at a higher dose. A therapeutically effective amount of one of the present compounds for treating emesis can then be determined by administering increasing amounts of such compound to a patient suffering from emesis until such time as the patient's symptoms of emesis are observed or are reported by the patient to be diminished or eliminated. A therapeutically effective amount of a compound for preventing emesis is determined in a similar fashion, except that the patient is treated prior to experiencing symptoms of emesis.

In a preferred embodiment, the present compounds and compositions selected for use in treating or preventing emesis for a particular subject or underlying condition have a therapeutic index of approximately 2 or greater. The therapeutic index is determined by dividing the dose at which adverse side effects occur by the dose at which efficacy for the condition is determined. A therapeutic index is preferably determined through the testing of a number of subjects.

Blood levels of the present compounds can be determined using routine biological and chemical assays and these blood levels can be matched to the route of administration. The blood level and route of administration giving the most desirable level of emesis relief can then be used to establish a therapeutically effective amount of a pharmaceutical composition comprising one of the present compounds for preventing and/or treating emesis.

Exemplary dosages in accordance with the teachings of the present invention for these compounds range from 0.0001 mg/kg to 60 mg/kg, though alternative dosages are contemplated as being within the scope of the present invention. Suitable dosages can be chosen by the treating physician by taking into account such factors as the size, weight, age, and sex of the patient, the physiological state of the patient, the severity of the condition for which the compound is being administered, the response to treatment, the type and quantity of other medications being given to the patient that might interact with the compound, either potentiating it or inhibiting it, and other pharmacokinetic considerations such as liver and kidney function.

Prevention of Emesis Example

A patient is determined to be in need of prevention of emesis as described above. A therapeutically effective amount of a compound of Formula (I) for preventing emesis is administered to the patient prior to the patient's experience of symptoms of emesis.

Treatment of Emesis Example

A patient is determined to be in need of treatment for emesis as described above. A therapeutically effective amount of a compound of Formula (I) for treating emesis is administered to the patient following the patient's experience of symptoms of emesis.

Pharmaceutical Compositions

Another aspect of the present invention is a method of preventing or treating emesis with a pharmaceutical composition that comprises: (1) an effective amount of a compound according to Formula I above (including salts and esters thereof); and (2) a pharmaceutically acceptable excipient.

A pharmaceutically acceptable excipient, including carriers, can be chosen from those generally known in the art including, but not limited to, inert solid diluents, aqueous solutions, liposomes, microspheres, or non-toxic organic solvents, depending on the route of administration. If desired, these pharmaceutical formulations can also contain preservatives and stabilizing agents and the like, for example substances such as, but not limited to, pharmaceutically acceptable excipients selected from the group consisting of wetting or emulsifying agents, pH buffering agents, human serum albumin, antioxidants, preservatives, bacteriostatic agents, dextrose, sucrose, trehalose, maltose, lecithin, glycine, sorbic acid, propylene glycol, polyethylene glycol, protamine sulfate, sodium chloride, or potassium chloride, mineral oil, vegetable oils and combinations thereof. Those skilled in the art will appreciate that other carriers also can be used.

Liquid compositions can also contain liquid phase excipients either in addition to or to the exclusion of water. Examples of such additional liquid phases are glycerin, vegetable oils such as cottonseed oil, organic esters such as ethyl oleate, and water-oil emulsions.

Formulations suitable for parenteral administration, such as, for example, by intravenous, intramuscular, intradermal, and subcutaneous routes, include aqueous and non-aqueous isotonic sterile injection solutions. These can contain antioxidants, buffers, preservatives, bacteriostatic agents, and solutes that render the formulation isotonic with the blood of the particular recipient. Alternatively, these formulations can be aqueous or non-aqueous sterile suspensions that can include suspending agents, thickening agents, solubilizers, stabilizers, and preservatives. The pharmaceutical compositions of the present invention can be formulated for administration by intravenous infusion, oral, topical, intraperitoneal, intravesical, transdermal, intranasal, rectal, vaginal, intramuscular, intradermal, subcutaneous and intrathecal routes.

Formulations of compound suitable for use in the present methods can be presented in unit-dose or multi-dose sealed containers, including in physical forms such as ampules or vials. The compositions can also be made into aerosol formations (i.e., they can be “nebulized”) to be administered via inhalation. Aerosol formulations can be placed into pressurized acceptable propellants, such as dichloromethane, propane, or nitrogen. Other suitable propellants are known in the art.

Although the present invention has been discussed in considerable detail with reference to certain preferred embodiments, other embodiments are possible. Therefore, the scope of the appended claims should not be limited to the description of preferred embodiments contained in this disclosure. All references cited herein are incorporated by reference to their entirety.

Groupings of alternative elements or embodiments of the invention disclosed herein are not to be construed as limitations. Each group member can be referred to and claimed individually or in any combination with other members of the group or other elements found herein. It is anticipated that one or more members of a group can be included in, or deleted from, a group.

1. A method of preventing or treating emesis, comprising the step of administering to a patient in need thereof a therapeutically effective amount of a pharmaceutical composition comprising a compound having the following formula (I): 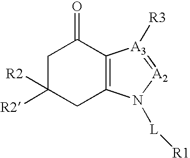
where:
(a) A₂ and A₃ are C or N;
(b) R₂ is hydrogen, alkyl, aralkyl, heteroaralkyl, aryl or heteroaryl;
(c) R_{2′} is hydrogen unless R₂ is alkyl, in which case R_{2′} is hydrogen or the same alkyl as R₂;
(d) R₃ is hydrogen, alkyl, aralky, heteroaralkyl, alkenyl, aralkenyl, heteroaralkenyl, aryl, heteroaryl, or does not exist when A₃ is N;
(e) L is a linker; and
(f) R1 has a formula selected from the group consisting of: 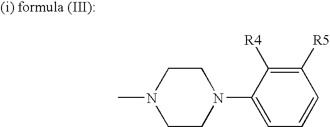
where:
(1) R₄ is hydrogen, alkyl, hydroxy, halo, alkoxy, cyano, methylthio;
(2) R₅ is hydrogen, alkyl, hydroxy, halo, alkoxy, trifluoromethyl, nitro, amino, aminocarbonyl, aminosulfonyl; and
(3) R₄ and R₅ can be taken together to form a 5 or 6 member aromatic or non-aromatic ring, which can contain from 0 to 3 heteroatoms selected from the group of N, O, or S of which the N may be further substituted if in a non-aromatic ring; 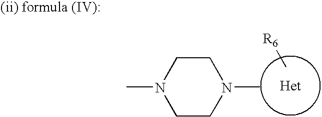
where the 6-member heterocyclic ring of formula (IV) is selected from the group consisting of a 2-pyridyl moiety, a 4-pyridyl moiety, a 2-pyrimidyl moiety, a 4-pyrimidyl moiety, a 2-pyrazinyl moiety, or a 2-triazinyl moiety; and 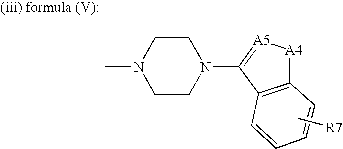
where:
(1) A₄ is N, O, or S, and when A₄ is N, it can be further substituted with Z, wherein Z is selected from the group consisting of alkyl, aralkyl, heteroaralky, and heteroalkyl;
(2) A₅ is C or N; and
(3) R₇ is selected from the group consisting of hydrogen, alkyl, NH₂, NHQ₁, NQ₁Q₂, OH, OQ₁, SQ₁, halo, nitro, cyano, and trifluoromethyl, and wherein Q₁ and Q₂ are selected from the group consisting of alkyl, aralkyl, heteroaralkyl, aryl, heteroaryl, alkanoyl, aroyl, aralkanoyl, heteroaralkanoyl, heteroaroyl, alkylsulfonyl, arylsulfonyl, heteroarylsulfonyl, aralkylsulfonyl, and heteroaralkylsulfonyl;
or a salt or ester thereof.

2. The method of claim 1, wherein the linker is selected from the group consisting of:
(a) a straight chain alkyl group having the formula —(CH₂)ₘ—, wherein m is an integer from 1 to 6; and
(b) an alkyl substituted hydrocarbyl moiety having the following formula (VI): 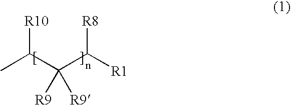
where:
(i)ₙ is 0, 1 or 2;
(ii) R8 and R10 are hydrogen, methyl or ethyl;
(iii) R9 and R9′ are both hydrogen, methyl or ethyl;
(iv) if n is 1 and R8 or R10 is methyl or ethyl, then R9 and R9′ are hydrogen;
(v) if n is 1 and R8 and R10 are hydrogen, then R9 and R9′ are methyl or ethyl; and
(vi) if n is 2, then R9 and R9′ are hydrogen and one or both of R8 and R10 are methyl or ethyl.

3. The method of claim 1, wherein:
(a) A₂ and A₃ are C;
(b) R₂ is hydrogen, alkyl, aralkyl, heteroaralkyl, aryl or heteroaryl; and
(c) R_{2′} and R₃ are hydrogen.

4. The method of claim 3, wherein R₂ is hydrogen.

5. The method of claim 4, wherein R1 is: 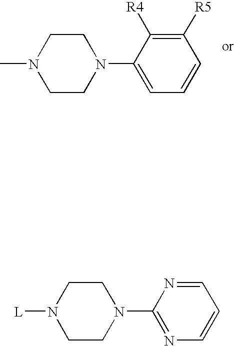
and
R₄ and R₅ are the same or independently hydrogen, alkyl, hydroxy, halo, alkoxy, trifluoromethyl, nitro, amino, aminocarbonyl, or aminosulfonyl.

6. The method of claim 1, wherein R₁ is a moiety selected from the group consisting of a m-trifluoromethylphenylpiperazinyl moiety, a m-chlorophenylpiperazinyl moiety, a o-methoxyphenylpiperazinyl moiety, a 1-naphthylpiperazinyl moiety, a 2-pyrimidylpiperazinyl moiety, and a 3-indazolylpiperazinyl moiety.

7. The method of claim 1, wherein R₆ is selected from the group consisting of a halo group, an alkyl group, a cyano group, a trifluoromethyl group, an alkoxy group, an amino group, an alkylamino group, or a dialkyamino group.

8. The method of claim 1, wherein the compound of formula (I) is selected from the group consisting of 1-{2-[4-(3-Trifluoromethylphenyl)piperazine-1-yl]ethyl}-1,5,6,7-tetrahydroindol-4-one; 1-{3-[4-(3-Trifluoromethylphenyl)piperazin-1-yl]propyl}-1,5,6,7-tetrahydroindol-4-one; 1-{4-[4-(3-Trifluoromethylphenyl)piperazin-1-yl]butyl}-1,5,6,7-tetrahydroindol-4-one; 1-{2-[4-(3-Chlorophenyl)piperazin-1-yl]ethyl}-1,5,6,7-tetrahydroindol-4-one; 1-{4-[4-(3-Chlorophenyl)piperazin-1-yl]butyl}-1,5,6,7-tetrahydroindol-4-one; 1-{2-[4-(2-Methoxyphenyl)piperazin-1-yl]ethyl}-1,5,6,7-tetrahydroindol-4-one; 1-{3-[4-(2-Methoxyphenyl)piperazine-1-yl]propyl}-1,5,6,7-tetrahydroindol-4-one; 1-{4-[4-(2-Methoxyphenyl)piperazin-1-yl]butyl}-1,5,6,7-tetrahydroindol-4-one; 1-{2-[4-(2-Pyrimidyl)piperazine-1-yl] ethyl}-1,5,6,7-tetrahydroindol-4-one; 1-{3-[4-(2-Pyrimidyl)piperazin-1-yl]propyl}-1,5,6,7-tetrahydroindol-4-one; 1-{4-[4-(2-Pyrimidyl)piperazin-1-yl]butyl}-1,5,6,7-tetrahydroindol-4-one; 1-{2-[4-(1-Naphthyl)piperazin-1-yl] ethyl}-1,5,6,7-tetrahydroindol-4-one; 1-{3-[4-(1-Naphthyl)piperazin-1-yl]propyl}-1,5,6,7-tetrahydroindol-4-one; 1-{4-[4-(1-Naphthyl)piperazin-1-yl]butyl}-1,5,6,7-tetrahydroindol-4-one; 1-{2-[4-(3-Indazolyl)piperazin-1-yl] ethyl}-1,5,6,7-tetrahydroindol-4-one; 1-{3-[4-(3-Indazolyl)piperazin-1-yl]propyl}-1,5,6,7-tetrahydroindol-4-one; and 1-{4-[4-(3-Indazolyl)piperazin-1-yl]butyl}-1,5,6,7-tetrahydroindol-4-one.

9. The method of claim 1, wherein the composition comprises a pharmaceutically acceptable excipient in combination with the compound of formula (I).

10. The method of claim 1, wherein the therapeutic dose is administered by an administrative route selected from the group consisting of intravenous infusion, oral, topical, intraperitoneal, intravesical, transdermal, nasal, rectal, vaginal, intramuscular, intradermal, subcutaneous and intrathecal routes.

11. The method of claim 1, wherein the therapeutically effective amount of the compound of formula (I) is in the range of 0.0001 mg/kg to 60 mg/kg.

12. The method of claim 1, wherein the therapeutically effective amount of the compound of formula (I) is administered to the patient after the onset of symptoms of emesis.

13. The method of claim 1, wherein the therapeutically effective amount of the compound of formula (I) is administered to the patient prior to the onset of symptoms of emesis.

14. The method of claim 1, wherein the therapeutically effective amount of the compound of formula (I) is administered to the patient prior to the administration of a chemotherapeutic agent.

15. The method of claim 14, wherein the chemotherapeutic agent is selected from the group consisting of cisplatin, cyclophosphamide, dacarbazine (DTIC), dactinomycin, mechlorethamine (nitrogen mustard), streptozocin, cyclophosphamide, carmustine (BCNU), lomustine (CCNU), doxorubicin (adriamycin), daunorubicin, procarbazine, mitomycin, cytarabine, etoposide, methotrexate, 5-fluorouracil, vinblastine, vincristine, bleomycin, paclitaxel and chlorambucil.

16. The method of claim 1, wherein the therapeutically effective amount of the compound of formula (I) is administered to the patient prior to the administration of an agent selected from the group consisting of an alpha-2 adrenoceptor antagonist and a type IV cyclic nucleotide phosphodiesterase inhibitor.

17. The method of claim 1, wherein the therapeutically effective amount of the compound of formula (I) is administered to the patient prior to the administration of radiation to the patient.

18. The method of claim 1, wherein the therapeutically effective amount of the compound of formula (I) is administered in combination with a chemotherapeutic agent.
